(19) United States
(12) Patent Application Publication
(10) Pub. No.: US 2005/0249679 A1
(43) Pub. Date: Nov. 10, 2005

(54) Compositions for accelerated tooth-whitening

(76) Inventors: Ryan B. Cameron, Somerset, NJ (US); Yelloji R. Mirajkar, Piscataway, NJ (US); Ravi Subramanyam, Belle Mead, NJ (US)

Correspondence Address: HARNESS, DICKEY, & PIERCE, P.L.C, 7700 BONHOMME, STE 400, ST. LOUIS, MO, 63105, US

(21) Appl. No.: 10/842,153

(22) Filed: May 10, 2004

(51) Int. Cl. A61K 7/20

(52) U.S. Cl. 424/53

(57) ABSTRACT

Dual-component tooth-whitening systems are disclosed comprising a peroxygen whitening agent in the first component and a transition metal catalyst along with an alkaline compound in the second component. Also disclosed are tooth-whitening methods based upon the systems.

### FIELD

This application relates generally to tooth-whitening compositions and, more particularly, to tooth-whitening compositions comprising a peroxygen compound, a transition metal catalyst and an alkaline compound and to methods for using the compositions.

### BACKGROUND

Tooth-whitening compositions are currently available and such compositions generally contain a peroxygen compound such as hydrogen peroxide or urea hydrogen peroxide (carbamide peroxide). Although such compositions can whiten stained teeth, it is generally considered that these compositions have a slow bleaching effect. Thus, there remains a need for new tooth-whitening compositions that produce a more rapid whitening of the teeth.

### SUMMARY

Accordingly, the present inventors have succeeded in devising compositions and methods for accelerating the whitening of teeth. The tooth-whitening compositions contain a peroxygen compound which is combined prior to use, with activating agents including a transition metal catalyst and an alkaline compound prior to use. The alkaline compound increases the pH of the tooth-whitening composition to a pH of 8.0 or greater. The transition metal catalyst and the increase in pH act synergistically to increase the tooth-whitening activity of the composition and thereby produce a more rapid whitening upon application to the teeth.

Thus, in various embodiments, the present invention can involve a dual component tooth-whitening system. The system can comprise a first component comprising a peroxygen whitening agent and a second activator component comprising one or more transition metal catalysts and one or more alkaline compounds. Upon mixing the first and second components a tooth-whitening composition having a pH of about 8.0 or greater is formed. The second activator component can be one composition comprising one or more transition metal catalysts and one or more alkaline compounds or multiple compositions of which at least one of the compositions comprises one or more transition metal catalyst and at least one other of the compositions comprises one or more alkaline compounds.

In various embodiments, the present invention can also involve a tooth-whitening composition. The composition can comprise a peroxygen whitening agent, one or more transition metal catalysts and one or more alkaline compounds. The pH of the composition can be 8.0 or greater. The tooth-whitening composition is suitable for application to the teeth in an effective amount to produce a tooth-whitening effect.

In various other embodiments, the present invention can also involve methods for whitening a tooth. As used herein, the term “tooth” in the singular form is intended to include the plural (teeth). The tooth-whitening method can comprise combining a composition comprising a peroxygen whitening agent with one or more transition metal catalysts and one or more alkaline compounds to form a tooth-whitening composition having a pH of about 8.0 or greater. The tooth-whitening composition is applied to a tooth to produce whitening of the tooth.

In various embodiments, the present invention can involve methods of tooth-whitening comprising applying to the surface of a tooth, a tooth-whitening composition which can comprise a peroxygen whitening agent, one or more transition metal catalysts and one or more alkaline compounds which produce a pH of the composition at 8.0 or greater.

In various embodiments, the present invention can also involve methods for enhancing the whitening activity of a tooth-whitening composition. The methods comprise providing a tooth-whitening composition comprising a peroxygen whitening agent and combining with the tooth-whitening composition, a metal catalyst and an alkaline compound. The composition thus formed has a pH of about 8.0 or greater and an enhanced tooth-whitening activity.

In various aspects of the present invention the peroxygen whitening agent can be one or more of a peroxide, a perborate, a percarbonate, a persulfate, a perphosphate, a persilicate, or a peroxyacid. The transition metal catalyst can be iron, cobalt nickel, copper, zinc, manganese, chromium, salts thereof or stabilized chelates thereof. The alkaline compound can be sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate, urea, monoethanolamine, diethanolamine, triethanolamine, mono(iso)propanolamine, di(iso)propanolamine, tri(iso)propanolamine, 2-amino-2-methylpropanol, 2-amino-2-methyl-1,3-propanediol, 2-amino-2-ethyl-1,3-propanediol, tris(hydroxymethyl)aminomethane, N,N,N′N′-tetrakis(2-hydroxypropyl)ethylenediamine, di(2-ethylhexyl)amine, triamylamine, dodecylamine, morpholine or combinations thereof.

In various embodiments, the peroxygen whitening agent can be hydrogen peroxide or urea hydrogen peroxide, the transition metal catalyst can be manganese gluconate salt, and the alkaline compound can be sodium bicarbonate.

It is also contemplated that in various embodiments, the one or more transition metal catalyst and the one or more alkaline compounds can be one or more compounds each of which serves as both a transition metal catalyst and an alkaline compound. For example, in various embodiments, the metal catalyst and the alkaline compound can be in the form of a salt, such as manganese bicarbonate.

### DETAILED DESCRIPTION

The present invention, in various embodiments, can involve methods and compositions for accelerating the whitening of teeth. The tooth-whitening compositions contain a peroxygen compound which is combined prior to use, with activating agents including one or more transition metal catalysts and one or more alkaline compounds prior to use.

The peroxygen compound can be any of a variety of peroxide-based bleaching agents, which deliver a hydrogen peroxide ion or an organic peroxide ion. Such compound include, for example, hydrogen peroxide, organic peroxide compounds, hydrogen peroxide generating compounds, organic peroxide generating compounds and combinations thereof.

Organic peroxide compounds include, for example, urea hydrogen peroxide (carbamide peroxide), glyceryl hydrogen peroxide as well as groups of peroxides classified according to the number and kind of organic functional groups attached to the oxygen atoms, such as, for example, alkyl hydrogen peroxide (R—O—O—H), dialkyl hydrogen peroxide (R—O—O—R′) peroxy acids (RCO—O—O—H), peroxy esters (RCO—OOR′), and diacyl peroxides (R—CO—O—O—CO—R′). Among such peroxides used in dental whitening are the diacyl peroxide, benzoyl peroxide and the peroxy acid monoperoxyphthalate.

In various embodiments, the peroxygen compound can also be a hydrogen peroxide generating compound such as, for example, alkali metal and alkaline-earth persulfate, dipersulfate, percarbonate, perphosphate, perborate, and persilicate salts such as, for example, sodium persulfate, sodium dipersulfate, sodium percarbonate, sodium perphosphate, sodium perborate, sodium persilicate, potassium persulfate potassium dipersulfate, potassium percarbonate, potassium perphosphate, potassium perborate, potassium persilicate, lithium dipersulfate, lithium percarbonate, lithium perphosphate, lithium perborate, lithium persilicate, calcium persulfate, calcium dipersulfate, calcium percarbonate, calcium perphosphate, calcium perborate, calcium persilicate, barium persulfate, barium dipersulfate, barium percarbonate, barium perphosphate, barium perborate, barium persilicate, magnesium persulfate, magnesium dipersulfate, magnesium percarbonate, magnesium perphosphate, magnesium perborate, and magnesium persilicate salts as well as sodium peroxide, potassium peroxide, lithium peroxide, calcium peroxide, barium peroxide and magnesium peroxide and combinations of any of the above compounds.

The peroxygen compound can also be any one or more of a peroxide, a perborate, a percarbonate, a persulfate, a perphosphate, a persilicate, or a peroxyacid.

In various embodiments, the peroxygen compound can be hydrogen peroxide or urea hydrogen peroxide.

The concentration of the peroxygen compound can be from about 0.1% (w/w) to about 30% (w/w), from about 0.5% (w/w) to about 25% (w/w), from about 1% (w/w) to about 20% (w/w), from about 2% (w/w) to about 10% (w/w) or from about 3% (w/w), about 3.5% (w/w) or about 4% (w/w) to about 7% (w/w) or about 10% (w/w).

The transition metal catalyst can comprise any of the stable transition elements in groups 3 through 12 of the periodic table including, for example, cadmium, chromium, cobalt, copper, gold, hafnium, iridium, iron, lutetium, manganese, mercury, molybdenum, nickel, niobium, osmium, palladium, platinum, rhenium, rhodium, ruthenium, scandium, silver, tantalum, titanium, tungsten, vanadium, yttrium, zinc or zirconium. In particular, the transition metal catalyst can comprise iron, cobalt nickel, copper, zinc, manganese, or chromium. In various embodiments, the transition metal catalyst can be iron, cobalt, nickel, copper, zinc, manganese or chromium and, in particular embodiments, the transitional metal catalyst can be manganese.

The transition metal catalyst can be in the form of a salt or a stabilized chelate. Salts of transition metal of the present invention, can be inorganic salts such as, for example, fluoride, chloride, bromide, iodide, nitrate, phosphate, sulfate salts and the like. In various embodiments, the salts can be manganese (II) difluoride, manganese (II) chloride, manganese(II) bromide, manganese (II) iodide, manganese (II) nitrate, manganese (II) phosphate, manganese (II) sulfate and the like. The transition metal salts of the present invention can also be in the form of organic salts such as, for example, carboxylic acid salts having two or more carbons. Such carboxylic acid salts can be, for example, salts of mono-carboxylic acid such as, for example, salts of acetic acid, propionic acid, butyric acid, valeric acid, caproic acid, heptanoic acid, caprylic acid and the like; salts of dicarboxylic acid such as, for example, salts of oxalic acid, malonic acid, succinic acid, adipic acid, and the like; or salts of hydroxy carboxylic acids such as, for example, salts of glycolic acid, lactic acid, malic acid, tartaric acid, citric acid, hexonoic hydroxy acids such as gluconic acid, gulonic acid, idonic acids such a glucuronic acid, galacturonic acid and mannuronic acid, saccharic acid, isosaccharic acid, heptonic hydroxy acids such as glucoheptanoic acid and combinations thereof. In various embodiments, the transition metal catalyst is manganese (II) gluconic acid.

In various embodiments, the transition metal catalyst can also be in the form of a complex of the transition metal and a multidentate ligand from a complexing agent such as is disclosed in U.S. Pat. No. 4,728,455 which is incorporated herein by reference. Such multidentate ligands chelate and stabilize the transition metal. The multidentate ligands can be supplied by a hydroxy carboxylic acid having at least 5 carbon atoms, such as, for example, hexonic hydroxy acids such as gluconic acid, gulonic acid, idonic acids such as glucuronic acid, galacturonic acid and mannuronic acid, heptonic hydroxy acids such as glucoheptanoic acid and sugars such as saccharic acid and isosaccharic acid. In various embodiments, the transition metal catalyst can be manganese in the form of manganese (III) gluconate as noted above. It is also possible for sorbitol and glycerol which are often present in dentifrice compositions, to chelate and stabilize the manganese as a transition metal catalyst in the form of manganese (III).

Other useful transition metal complexes and, in particular, manganese coordination complex compounds of the present invention include manganese complexes of the formula:
LnMnX
wherein Mn is manganese in the +3 or +4 oxidation state; n and m are integers from 1 to 4; X represents a coordination or a bridging species that coordinates with the manganese and is selected from H₂O, OH⁻, O₂⁻, SH⁻, and alkyl and aryl groups having 1 to 20 carbon atoms and L is a ligand having at least 2 nitrogen, phosphorus, oxygen or sulfur atoms coordinating with the manganese. Examples of ligands suitable for the formation of the manganese complexes of the formula are more fully described in U.S. Pat. No. 5,194,416, which is incorporated herein by reference. Preferred examples of L in the formula above include: 1,4,7-triazacyclononane, 1,4,7-triazacyclodecane, 1,4,8-triazacycloundecane, 1,5,9-triazacyclodecane, 1,4,7-trimethyl-1,4,7-triazacyclononane, 1,4,7-trimethyl-1,4,7-triazacyclodecane, 1,4,8-trimethyl-1,4,8-triazacycloundecane, 1,5,9-trimethyl-1,5,9-triazacyclododecane, tris(pyridin-2-yl)methane, tris(pyrazol-1-yl)methane, tris(imidazol-2-yl)methane, tris(pyridin-2-yl)borate, tris(imidazol-2-yl)phosphine, 1,1,1-tris(methylamino)ethane, Bis(pyridin-2-yl-methyl)amine, Bis(triazol-1-yl-methyl)amine and Bis(imidazol-2-yl-methyl)amine.

The transition metal catalyst can be at a concentration of from about 0.01 ppm to about 100,000 ppm, from about 0.1 ppm to about 10,000 ppm, from about 1 ppm to about 1,000 ppm, from about 2.5 ppm to about 500 ppm, or from about 6.25 or about 12.5 to about 125 ppm in the tooth-whitening composition by weight.

The alkaline compound increases the pH of the tooth-whitening composition to a pH of about 8.0 or greater, about 8.5 or greater, about 9.0 or greater, about 9.5 or greater or about 10.0 or greater.

The alkaline compound can be any of a number of compounds capable of increasing the pH of an aqueous solution such as, for example, an alkali metal, ammonium or alkaline-earth metal compound including, for example, sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate or combinations thereof; and organic amines such as urea, alkanolamines such as monoethanolamine, diethanolamine, triethanolamine, mono(iso)propanolamine, di(iso)propanolamine, tri(iso)propanolamine or 2-amino-2-methylpropanol; alkanediolamines such as 2-amino-2-methyl-1,3-propanediol or 2-amino-2-ethyl-1,3-propanediol; alkanepolyamines such as tris(hydroxymethyl)aminomethane or N,N,N′N′-tetrakis(2-hydroxypropyl)ethylenediamine; alkylamines such as di(2-ethylhexyl)amine, triamylamine or dodecylamine; or amino ethers such as morpholine.

The alkaline compound can be at a concentration of from about 0.1% (w/w) to about 30% (w/w), from about 0.2% (w/w) to about 10% (w/w), from about 0.5% (w/w) or about 0.75% (w/w) to about 2.5% (w/w) or about 5% (w/w) in the tooth-whitening composition. In various embodiments, the alkaline compound can be sodium bicarbonate and after mixing with the composition containing the peroxygen compound, the sodium bicarbonate can be at a concentration of about 2.5% (w/w) or about 5% (w/w) in the tooth-whitening composition.

The transition metal catalyst and the alkaline compound can be in separate compositions or in one composition and the peroxygen compound can be in a composition separate from both the transition metal catalyst and the alkaline compound in order to prevent destabilization of the peroxygen compound. This allows the composition comprising the peroxygen compound to remain in a shelf-stable condition. Prior to use, the transition metal catalyst and the alkaline compound are combined with the peroxygen compound.

The transition metal catalyst and the alkaline compound which increases pH of the composition act synergistically to increase the tooth-whitening activity of the composition and thereby produce a more rapid whitening upon application to the teeth. By acting synergistically it is meant that the whitening effect produced by the combination of the transition metal catalyst and the alkaline compound is greater the effect produced by either of the activators alone and, in various embodiments, the effect produced by the combination is greater than the sum of the effects produced by the activators alone. This can produce an acceleration of the whitening effect and, in various embodiments, it can allow the use of lower concentrations of the peroxygen compound.

The amount of transition metal catalyst, and in particular, the amount of manganese catalyst present in the second component of the two phase tooth-whitening composition of the present invention can vary dependent upon the amount of peroxygen compound incorporated in the first component. When the whitening oral composition is to be used by trained professionals and the first component can contain relatively high concentrations of a peroxygen compound, for example, 5% (w/w) to 35% (w/w), the amount of manganese catalyst compound incorporated in the second component will range between 0.1% (w/w) to 3% (w/w) and preferably between 0.25% (w/w) to 1.75% (w/w). For home use, compositions in which the concentration range of peroxygen compound in the first oral composition component is between about 0.1% to about 3.0% (w/w), lower concentrations of the manganese catalyst can be included in the second component, for example, from about 0.001% (w/w) to about 0.3% (w/w) or from about 0.0025% (w/w) to about 0.15% (w/w).

The amount of alkaline compound present in the second component of the two phase tooth-whitening composition will depend upon the various components present in the compositions and the concentration will be sufficient to increase the pH of the tooth-whitening composition to a value of 8.0 or greater, 8.5 or greater, 9.0 or greater, 9.5 or greater or 10.0 or greater.

In various embodiments, the compositions of the present invention can also contain a vehicle which can include water and a humectant such as glycerin, propylene glycol, polyethylene glycol, or any mixture thereof. In various embodiments, a mixture of glycerin and polyethylene glycol can be used as humectants.

The proportion of vehicle in the compositions of the present invention can be from about 20% (w/w) to about 95% (w/w), from about 40% (w/w) to about 80% (w/w), or from about 50% (w/w) to about 65% (w/w).

In certain embodiments of multiple component systems of the present invention, such as in dual-component dentifrices, the first and second components can have substantially the same vehicle and substantially the same percent of vehicle present in the compositions, whereas in other embodiments the first and second components can have either or both of different vehicles and different percents of vehicle present in the compositions.

In various embodiments, one or more surfactants can be included in the compositions of the present invention. Such surfactants can include salts of the higher alkyl sulfates and alkyl phosphates having about 8 to about 18 carbon atoms in the alkyl group such as sodium lauryl sulfate and sodium lauryl phosphate, sodium lauryl sulfoacetate, salts of sulfonated monoglycerides of higher fatty acids, such as sodium coconut monoglyceride sulfonate or other suitable sulfonated monoglycerides of a fatty acids of about 10 to about 18 carbon atoms; salts of amides of higher fatty acids, e.g., about 12 to about 16 carbon atom acids, with lower aliphatic amino acids, such as sodium-N-methyl-N-palmitoyl tauride, sodium N-lauroyl-, N-myristoyl- and N-palmitoyl sarcosinates; salts of the esters of such fatty acids with isothionic acid or with glycerol monosulfate, such as the sodium salt of monosulfated monoglyceride of hydrogenated coconut oil fatty acids.

In various embodiments, the surfactant can be present in the compositions of the present invention at a concentration of from about 0.5% (w/w) to about 3.0% (w/w) and preferably about 1.0% (w/w) to about 2.0% (w/w).

Polishing agents can also be incorporated into the compositions in various embodiments of the present invention. Such polishing agents can be siliceous materials, such as silica, which have a mean particle size up to about 20 microns. In various embodiments, the silica can be a precipitated amorphous hydrated silica, such as Sorbosil silicates, for example, Sorbosil AC-35 (INEOS Silicas Ltd., Warrington, UK), or the Zeodent® silicates, for example Zeodent® 115 from J.M. Huber Company (Edison, N.J.). Other polishing agents can also be used, including sodium metaphosphate, potassium metaphosphate, tricalcium phosphate, calcium phosphate dihydrate, anhydrous dicalcium phosphate, calcium pyrophosphate, magnesium orthophosphate, trimagnesium phosphate, alumina trihydrate, aluminum silicate, zirconium silicate, calcined alumina and bentonite.

The polishing agent can be present in the dentifrice compositions of the present invention at a concentration of from about 10% (w/w) to about 30% (w/w) or from about 15% (w/w) to about 30% (w/w).

Thickeners can also be included in the compositions of the present invention. Inorganic thickeners can include fumed silicas such as Cabosil available from Cabot Corporation (Boston, Mass.), and thickening silicas such as Sylox 15, which is available from W. R. Grace (Columbia, Md.). Organic thickeners such as natural and synthetic gums and colloids can also be included in the compositions of the present invention. Examples of such thickeners include carrageenan (Irish moss), xanthan gum and sodium carboxymethyl cellulose, starch, polyvinylpyrrolidone, hydroxyethylpropylcellulose, hydroxybutyl methyl cellulose, hydroxypropyl methyl cellulose and hydroxyethyl cellulose.

In various embodiments, the inorganic or organic thickener can be present in the compositions of the present invention at a concentration of from about 0.05% (w/w) to about 2% (w/w) or from about 0.1% (w/w) to about 1.5% (w/w).

The compositions in various embodiments of the present invention, can also contain fluoride salts. Fluoride-providing salts having anti-caries efficacy can also be incorporated in the oral compositions of the present invention and are characterized by their ability to release fluoride ions in water. It is preferable to employ a water-soluble salt fluoride providing fluoride ion at a concentration of from about 1 to about 5,000 ppm, from about 10 to about 5,000 ppm, from about 100 to about 2500 ppm or from about 1000 to about 1500 ppm of fluoride ion. Among these materials are water-soluble alkali metal salts, for example, sodium fluoride, potassium fluoride, sodium monofluorophosphate and sodium fluorosilicate. Sodium fluoride and sodium monofluorophosphate are preferred fluoride-providing salts.

Salts having anti-tartar efficacy, including water soluble salts, such as dialkali or tetra-alkali metal pyrophosphate salts such as Na₄P₂O₇ (TSPP) K₄PO₇, Na₂ K_{2,}P₂O₇, Na₂H₂P₂O₇ and K₂H₂P₂O₇, long chain polyphosphate such as sodium hexametaphosphate and cyclic phosphates such as sodium trimetaphosphate as well as alkali metal tripolyphosphates such as sodium tripolyphosphate (STPP) and potassium tripolyphosphate may be incorporated in the dentifrice compositions of the present invention preferably at a concentration of about 0.5 to about 8.0% by weight.

In various embodiments of the present invention, one or more colorants can be included in the compositions. For dual-component systems, contrasting colors can be included in the first and second components and in various aspects of such embodiments, the system can be in the form of a striped dentifrice product. The colorants are pharmacologically and physiologically non-toxic when used in the appropriate amounts. Colorants used in the practice of the present invention include both pigments and dyes.

Pigments can include non-toxic, water insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green No. 1 lake, FD&C Blue No. 2 lake, FD&C Red No. 30 lake and FD&C Yellow No. 15 lake and mixtures thereof. The pigments can have a particle size in the range of from about 5 microns to about 1000 microns or from about 250 microns to about 500 microns. The concentration of such pigments can be from about 0.5% (w/w) to about 3% (w/w).

In certain embodiments, dyes can be distributed uniformly throughout the compositions or throughout one or more components of the dual component systems of the present invention. The dyes are generally food color additives presently certified under the Food Drug & Cosmetic Act for use in food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-N-ethyl-N-p-sulfobenzyl)-.DELTA.-3,5-cyclohexadienimine], FD&C Blue No. 1 (disodium salt of dibenzyldiethyl-diaminotriphenylcarbinol trisulfonic acid anhydride), FD&C Blue No. 2 (sodium salt of disulfonic acid of indigotin) and mixtures thereof in various proportions. The concentration of the dye for the most effective result in the present invention is present in the dentifrice composition in an amount from about 0.0005% (w/w) to about 2% (w/w).

In various embodiments, the colorant can be a pigment such as TiO₂ distributed in a first component of a dual component system and another colorant can be distributed throughout the vehicle of the second component. The colorant, if present in the second component, can be a dye of a different color than the pigment included in the first component.

Flavoring materials can also be included in the compositions of the present invention. Any suitable flavoring or sweetening material can be used. Examples of suitable flavoring constituents are flavoring oils, e.g., oils of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, sodium cyclamate, perillartine, and sodium saccharin. Suitably, flavor and sweetening agents can together comprise from about 0.01% (w/w) to about 5% (w/w) or more of the compositions of the present invention.

Various other materials can be included in the compositions of the present invention. Non-limiting examples include preservatives, silicones and chlorophyll compounds, vitamins such as vitamins B6, B12, C, E and K, antibacterial agents such as chlorohexidene, halogenated diphenyl ethers such as triclosan, desensitizing agents such as potassium nitrate and potassium citrate and mixtures thereof. These substances can be included in amounts which do not substantially adversely affect the properties and characteristics desired, and are selected and used in proper amounts, depending upon the particular type of component involved.

In various embodiments, a peroxygen compound containing dentifrice paste or gel component of the present invention can be prepared. Humectants such as, for example, propylene glycol, glycerin, polyethylene glycol ingredients, sweetener and water can be dispersed in a conventional mixer until the mixture becomes a homogeneous gel phase. Into the gel phase a pigment can be added such as TiO₂ and any tartar control agents such as tetrasodium pyrophosphate or sodium tripolyphosphate or both and fluoride anti-caries agents such as sodium monofluorophosphate. These ingredients can be mixed until a homogeneous phase is obtained. Thereafter the thickener, polishing agent, peroxygen compound, flavor and surfactant ingredients are added and the ingredients mixed at high speed under vacuum of from about 20 to about 100 mm Hg. The resultant product is a homogeneous, semi-solid, extrudable paste product.

A second component containing a transition metal catalyst and an alkaline compound can be prepared in a manner similar to that described above for the peroxygen-containing composition. The transition metal catalyst and alkaline compound are included and, optionally, a dye can be included in the initial mixture of humectants and sweetener and TiO₂. The peroxygen compound is omitted from the second component.

To prepare a rinse composition the various ingredients are mixed together in water in a conventional manner.

In packaging the oral composition of the present invention for sale, any convenient means for effecting the separation of the peroxygen compound from the activator components before use can be utilized. For example in the packaging of dentifrice components, a single container can be compartmentalized so that the peroxygen containing dentifrice component and the activator containing component are housed in separate compartments and are dispensed simultaneously for common application to a toothbrush and not admixed until applied to the teeth. Alternatively, the peroxygen containing component and the activator containing component can be housed in separate containers from which the respective phases are dispensed for admixture just prior to use.

The following examples are further illustrative of the present invention, but it is understood that the invention is not limited thereto. All amounts and proportions referred to herein and the appended claims are by weight unless otherwise indicated.

### EXAMPLE 1

This example illustrates the degradation of peroxygen compounds in the presence of transition metal catalysts and sodium bicarbonate.

Both oxidative and degradative pathways of hydrogen peroxide decomposition involve the breakdown of peroxide yielding water and molecular oxygen. Because two H₂O₂ molecules yield one O₂ molecule, the rate of decomposition H₂O₂ is twice the rate of oxygen evolved. A gasometric system was set up to measure the rate of oxygen evolved as a function of time.

Temperature was maintained at 37 C using a water bath heated by a stirring hot plate. The reagent mix was added to a mixing vessel closed with a rubber septum and connected to a reservoir of water to be displaced by evolved gas. The displaced water was measured in a measuring cylinder and the time was recorded using a timer. A brisk rate of stirring was maintained throughout the experiment. The initial rate of gas evolution was measured in the early stages of the reaction (1-1.5 min).

Solutions were prepared as follows. Buffer solution (pH 8) having ionic strength of 0.1M was made from 0.68 g monobasic sodium phosphate (NaH₂PO₄) and 11.47 g dibasic sodium phosphate (Na₂HPO₄) dissolved in 1 L of deionized water at room temperature. A pH 8.8 solution was made by dropwise addition of 6M sodium hydroxide solution to the pH 8 buffer solution while stirring and measuring the pH. Stock manganese gluconate (formula weight 445.3 g/mol) solution containing 250 ppm manganese gluconate was prepared by dissolving 62.5 mg manganese gluconate into 250 ml deionized water or specified buffer solution. Test solutions containing various concentrations of NaHCO₃, hydrogen peroxide (H₂O₂), urea hydrogen peroxide, and manganese gluconate were prepared from stock solutions of NaHCO₃, H₂O₂, hydrogen peroxide, and manganese gluconate.

Ten ml solutions (pH 8.8) were tested containing various concentrations of NaHCO₃, hydrogen peroxide (H₂O₂), urea hydrogen peroxide, and manganese gluconate. As shown in Table 1, the presence of 5% sodium bicarbonate at a pH of 8.8, increased the rate of peroxide degradation and the additional presence of manganese gluconate produced a substantial further increase in peroxide degradation. Values shown in the table are the average of two measurements.

TABLE 1

| | | Manganese gluconate | Initial Rate |
|---|---|---|---|
| H₂O₂ (%) | NaHCO₃ (%) | (ppm) | (min⁻¹) |
| 1 | — | — | — |
| 1 | — | 12.5 | — |
| 1 | 5 | — | 4.8 |
| 1 | 5 | 12.5 | 12.2 |
| 1 | 5 | 333 | 47.0 |

Table 2 shows similar results for urea hydrogen peroxide. Values in the table are the average of two experiments.

TABLE 2

| Urea Hydrogen | | Manganese gluconate | Initial Rate |
|---|---|---|---|
| Peroxide (%) | NaHCO₃ (%) | (ppm) | (min⁻¹) |
| 3 | — | 12.5 | 1.5 |
| 3 | 5 | — | 4.8 |
| 3 | 5 | 12.5 | 17.6 |
| 3 | 5 | 333 | 47.6 |

### EXAMPLE 2

This example illustrates the bleaching of indicator dyes by peroxygen compounds in the presence of transition the presence of transition metal catalysts and sodium bicarbonate.

The bleaching effect of peroxygen compounds in the presence of transition metal catalysts and sodium bicarbonate was tested using the dye Lissamine Green which is susceptible to bleaching by low concentrations of peroxygen compounds at room temperature. The rate of bleaching was measured spectraphotometrically by monitoring the rate of disappearance of the dye at a λₘₐₓ of 625 with time. Components were prepared in stock solutions and added to 3 ml cuvettes to form mixtures shown in the tables. Rate of dye degradation was expressed as the half-life (t_{1/2}) which was the time for the concentration of the dye to be bleached to half of its initial level. This was calculated from the initial rate of bleaching suring the first 1 to 1.5 min. Lissamine Green B was present at a concentration of 1.56×10⁻⁴ M.

As shown in Table 3, sodium bicarbonate in absence of manganese gluconate, modestly decreased the t_{1/2} indicated an increase in bleaching rate. Manganese gluconate in absence of sodium bicarbonate, produced a slight increase the t_{1/2} indicating a slight decrease in bleaching rate. In comparison to this, the combination of sodium bicarbonate at concentrations of 2.5% and 5% and manganese gluconate at concentrations of 6.25, 12.5 and 125 ppm substantially decreased t_{1/2} indicating a substantial increase in bleaching rate.

TABLE 3

| | | Manganese gluconate | |
|---|---|---|---|
| H₂O_{2 (%)} | NaHCO₃ (%) | (ppm) | t½ (min) |
| 0.01 | — | — | 12 |
| 0.01 | — | 125 | 14 |
| 0.01 | 5 | — | 7 |
| 0.01 | 5 | 12.5 | 0.3 |
| 0.01 | 5 | 125 | 0.6 |
| 0.01 | 2.5 | 6.25 | 1.9 |
| 0.01 | 2.5 | 12.5 | 1.9 |
| 0.01 | 2.5 | 125 | 1.1 |

Similar results were produced by urea hydrogen peroxide (Table 4).

TABLE 4

| Urea Hydrogen | | Manganese gluconate | |
|---|---|---|---|
| Peroxide (%) | NaHCO₃ (%) | (ppm) | t_{1/2} (min) |
| 0.03 | 5 | 12.5 | 0.3 |
| 0.03 | 5 | 125 | 0.4 |
| 0.01 | 2.5 | 12.5 | 2.7 |
| 0.01 | 2.5 | 125 | 1.1 |

The data show that the combination of aqueous sodium bicarbonate and manganese (II) gluconate produces a synergistic bleaching effect for the peroxygen compounds, hydrogen peroxide and urea hydrogen peroxide.

All references cited in this specification are hereby incorporated by reference. Any discussion of references cited herein is intended merely to summarize the assertions made by their authors and no admission is made that any reference or portion thereof constitutes relevant prior art. Applicants reserve the right to challenge the accuracy and pertinency of the cited references.

The description of the invention is merely exemplary in nature and, thus, variations that do not depart from the gist of the invention are intended to be within the scope of the invention. Such variations are not to be regarded as a departure from the spirit and scope of the invention.

1. A dual component tooth-whitening system comprising a first component comprising a peroxygen whitening agent and a second component comprising one or more transition metal catalysts and one or more alkaline compounds, wherein upon mixing the first and second components a tooth-whitening composition having a pH of about 8.0 or greater is formed.

2. A system according to claim 1, wherein the peroxygen whitening agent is selected from the group consisting peroxides, perborates, percarbonates, persulfates, perphosphates, persilicates, peroxyacids and combinations thereof.

3. A system according to claim 1, wherein the transition metal catalyst is selected from the group consisting of iron, cobalt, nickel, copper, zinc, manganese, chromium, salts thereof and stabilized chelates thereof.

4. A system according to claim 1, wherein the alkaline compound is selected from the group consisting of sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate, urea, monoethanolamine, diethanolamine, triethanolamine, mono(iso)propanolamine, di(iso)propanolamine, tri(iso)propanolamine, 2-amino-2-methylpropanol, 2-amino-2-methyl-1,3-propanediol, 2-amino-2-ethyl-1,3-propanediol, tris(hydroxymethyl)aminomethane, N,N,N′N′-tetrakis(2-hydroxypropyl)ethylenediamine, di(2-ethylhexyl)amine, triamylamine, dodecylamine, morpholine and combinations thereof.

5. A system according to claim 1, wherein the peroxygen whitening agent is hydrogen peroxide or urea hydrogen peroxide, the transition metal catalyst is manganese gluconate salt, and the alkaline compound is sodium bicarbonate.

6. A tooth-whitening composition comprising a peroxygen whitening agent, one or more transition metal catalysts and one or more alkaline compounds, wherein said composition has a pH of about 8.0 or greater.

7. A composition according to claim 6, wherein the peroxygen whitening agent is selected from the group consisting peroxides, perborates, percarbonates, persulfates, perphosphates, persilicates, peroxyacids and combinations thereof.

8. A composition according to claim 6, wherein the transition metal catalyst is selected from the group consisting of iron, cobalt, nickel, copper, zinc, manganese, chromium, salts thereof and stabilized chelates thereof.

9. A composition according to claim 6, wherein the alkaline compound is selected from the group consisting of sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate, urea, monoethanolamine, diethanolamine, triethanolamine, mono(iso)propanolamine, di(iso)propanolamine, tri(iso)propanolamine, 2-amino-2-methylpropanol, 2-amino-2-methyl-1,3-propanediol, 2-amino-2-ethyl-1,3-propanediol, tris(hydroxymethyl)aminomethane, N,N,N′N′-tetrakis(2-hydroxypropyl)ethylenediamine, di(2-ethylhexyl)amine, triamylamine, dodecylamine, morpholine and combinations thereof.

10. A composition according to claim 6, wherein the peroxygen whitening agent is hydrogen peroxide or urea hydrogen peroxide, the transition metal catalyst is manganese gluconate salt, and the alkaline compound is sodium bicarbonate.

11. A method for whitening a tooth, the method comprising combining a composition comprising a peroxygen whitening agent with one or more transition metal catalyst and one or more alkaline compound to form a tooth-whitening composition having a pH of about 8.0 or greater and applying said tooth-whitening composition to a tooth.

12. A method according to claim 11, wherein the peroxygen whitening agent is selected from the group consisting peroxides, perborates, percarbonates, persulfates, perphosphates, persilicates, peroxyacids and combinations thereof.

13. A method according to claim 11, wherein the transition metal catalyst is selected from the group consisting of iron, cobalt, nickel, copper, zinc, manganese, chromium, salts thereof and stabilized chelates thereof.

14. A method according to claim 11, wherein the alkaline compound is selected from the group consisting of sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate, urea, monoethanolamine, diethanolamine, triethanolamine, mono(iso)propanolamine, di(iso)propanolamine, tri(iso)propanolamine, 2-amino-2-methylpropanol, 2-amino-2-methyl-1,3-propanediol, 2-amino-2-ethyl-1,3-propanediol, tris(hydroxymethyl)aminomethane, N,N,N′N′-tetrakis(2-hydroxypropyl)ethylenediamine, di(2-ethylhexyl)amine, triamylamine, dodecylamine, morpholine and combinations thereof.

15. A method according to claim 11, wherein the peroxygen whitening agent is hydrogen peroxide or urea hydrogen peroxide, the transition metal catalyst is manganese gluconate salt, and the alkaline compound is sodium bicarbonate.

16. A method for whitening a tooth, the method comprising applying to the surface of a tooth, a tooth-whitening composition according to claim 6.

17. A method according to claim 16, wherein the peroxygen whitening agent is selected from the group consisting peroxides, perborates, percarbonates, persulfates, perphosphates, persilicates, peroxyacids and combinations thereof.

18. A method according to claim 16, wherein the transition metal catalyst is selected from the group consisting of iron, cobalt, nickel, copper, zinc, manganese, chromium, salts thereof and stabilized chelates thereof.

19. A method according to claim 16, wherein the alkaline compound is selected from the group consisting of sodium hydroxide, potassium hydroxide, ammonium hydroxide, calcium hydroxide, magnesium hydroxide, sodium carbonate, potassium carbonate, ammonium carbonate, calcium carbonate, magnesium carbonate, sodium bicarbonate, potassium bicarbonate, ammonium bicarbonate, calcium bicarbonate, magnesium bicarbonate, urea, monoethanolamine, diethanolamine, triethanolamine, mono(iso)propanolamine, di(iso)propanolamine, tri(iso)propanolamine, 2-amino-2-methylpropanol, 2-amino-2-methyl-1,3-propanediol, 2-amino-2-ethyl-1,3-propanediol, tris(hydroxymethyl)aminomethane, N,N,N′N′-tetrakis(2-hydroxypropyl)ethylenediamine, di(2-ethylhexyl)amine, triamylamine, dodecylamine, morpholine and combinations thereof.

20. A method according to claim 16, wherein the peroxygen whitening agent is hydrogen peroxide or urea hydrogen peroxide, the transition metal catalyst is manganese gluconate salt, and the alkaline compound is sodium bicarbonate.

21. A method for enhancing the whitening activity of a tooth-whitening composition, the method comprising providing a tooth-whitening composition comprising a peroxygen whitening agent and combining said tooth-whitening composition with one or more metal catalysts and one or more alkaline compounds to form a composition having a pH of about 8.0 or greater and an enhanced tooth-whitening activity.
